(19) United States
(12) Patent Application Publication
(10) Pub. No.: US 2014/0212981 A1
(43) Pub. Date: Jul. 31, 2014

(54) METHODS AND SYSTEMS FOR DETERMINING THE AMOUNT OF THIOPURINE METABOLITES IN A SAMPLE

(71) Applicant: Laboratory Corporation of America Holdings, Burlington, NC (US)

(72) Inventors: Russell Philip Grant, Chapel Hill, NC (US); Stacy Michelle Walker Dee, Yanceyville, NC (US)

(73) Assignee: Laboratory Corporation of America Holdings, Burlington, NC (US)

(21) Appl. No.: 14/135,118

(22) Filed: Dec. 19, 2013

Related U.S. Application Data
(60) Provisional application No. 61/739,427, filed on Dec. 19, 2012.

(51) Int. Cl. G01N 33/52

(52) U.S. Cl. CPC G01N 33/52
USPC 436/94; 436/98; 422/70

(57) ABSTRACT

Disclosed are methods and systems for the analysis of thiopurine drug metabolites in a sample using liquid chromatography/mass spectrometry.

### FIELD OF INVENTION

The present invention relates generally to methods and systems for determining the presence or amount of one or more drug metabolites in a sample. In some embodiments, the invention provides methods and systems for analyzing certain metabolites of thiopurine drugs in a sample using liquid chromatography and mass spectrometry.

### BACKGROUND

Thiopurines are compounds that may be used in the treatment of certain disorders, including Crohn's disease and irritable bowel syndrome (IBS). When thiopurines are administered to a human subject, the drug compounds are metabolized to yield various metabolites, some of which can be biologically active while others can be biologically inactive. Certain of these metabolites can be further phosphorylated in the subject.

Dosing of thiopurines can present certain difficulties. The presence or abundance of certain genetic polymorphisms can affect the subject's sensitivity or toxicity to the drug. This can occur because thiopurines can be metabolized via interactions with the enzyme thiopurine methyl transferase (TPMT), which can be up- or down-regulated depending on the subject's gene expression. Thus, in some instances, what may be an effective dose of a thiopurine drug for one subject, could be a sub-optimal dose or a toxic dose for another subject, depending on the presence or abundance of certain genetic polymorphisms.

Consequently, modulation of dosing during the course of treatment (or at least at the early stages of treatment) may be necessary to assess what is an effective, non-toxic dose for a certain subject. Therefore, there is a need for diagnostic tests that can effectively assess whether a subject is in need of modulation of thiopurine dosing during the course of treatment.

### SUMMARY OF THE INVENTION

In at least one aspect, the invention provides methods for determining the amount of thiopurine drug metabolites in a sample, the methods comprising: (a) providing a sample comprising one or more 6-thioguanine nucleotide compounds and one or more 6-methylmercaptopurine nucleotide compounds; (b 1) converting the one or more 6-thioguanine nucleotide compounds to 6-thioguanine or a 6-thioguanine hydrolysis product, and (b2) converting the one or more 6-methyl-mercaptopurine nucleotide compounds to 6-methylmercaptopurine or a 6-methylmercaptopurine hydrolysis product; (c1) chromatographically separating the 6-thioguanine or the 6-thioguanine hydrolysis product from other components in the converted sample using liquid chromatography, and (c2) chromatographically separating the 6-methylmercaptopurine or the 6-methylmercaptopurine hydrolysis product from other components in the converted sample using liquid chromatography; (d1) analyzing the chromatographically separated 6-thioguanine or the 6-thioguanine hydrolysis product by mass spectrometry to determine the amount of the one or more 6-thioguanine nucleotide compounds in the sample, and (d2) analyzing the chromatographically separated 6-methylmercaptopurine or the 6-methylmercaptopurine hydrolysis product by mass spectrometry to determine the amount of the one or more 6-methylmercaptopurine nucleotide compounds in the sample. In some embodiments, the sample originates from a biological fluid, such as whole blood. In some such embodiments, the method comprises determining a relative amount of the one or more 6-thioguanine nucleotide compounds and the one or more 6-methylmercaptopurine nucleotide compounds to red blood cells in the biological sample. In some such embodiments, the amounts of the one or more 6-thioguanine nucleotide compounds and the one or more 6-methylmercaptopurine nucleotide compounds are correlated to assess the efficacy or toxicity of treatment of a subject undergoing treatment with a thiopurine drug. In certain embodiments, the therapeutic range for the 6-thioguanine or the 6-thioguanine hydrolysis product is about 235-450 pmol (pico mole) per 8×10⁸ red blood cells. In certain embodiments, the toxic value for the 6-thioguanine or the 6-thioguanine hydrolysis product is greater than about 450 pmol per 8×10⁸ red blood cells. In certain embodiments, the therapeutic range for the 6-methylmercaptopurine or a 6-methylmercaptopurine hydrolysis product is less than about 5700 pmole per 8×10⁸ red blood cells. Also, in certain embodiments, the toxic value for the 6-methylmercaptopurine or a 6-methylmercaptopurine hydrolysis product is greater than about 5700 pmole per 8×10⁸ red blood cells. Further embodiments of these methods are described in detail below.

In another aspect, the invention provides methods for determining the amount of thiopurine drug metabolites in a sample, the methods comprising: (a) providing a sample comprising one or more 6-thioguanine nucleotide compounds; (b) converting the one or more 6-thioguanine nucleotide compounds to 6-thioguanine or a 6-thioguanine hydrolysis product; (c) chromatographically separating the 6-thioguanine or the 6-thioguanine hydrolysis product from other components in the converted sample using liquid chromatography; (d) analyzing the chromatographically separated 6-thioguanine or the 6-thioguanine hydrolysis product by mass spectrometry to determine the amount of the one or more 6-thioguanine nucleotide compounds in the sample. In some embodiments, the sample originates from a biological fluid, such as whole blood. In some such embodiments, the method comprises determining a relative amount of the one or more 6-thioguanine nucleotide compounds to red blood cells in the biological sample. In some such embodiments, the amount of the one or more 6-thioguanine nucleotide compounds is correlated to assess the efficacy or toxicity of treatment of a subject undergoing treatment with a thiopurine drug. In certain embodiments, the therapeutic range for the 6-thioguanine or the 6-thioguanine hydrolysis product is about 235-450 pmol per 8×10⁸ red blood cells. In certain embodiments, the toxic value for the 6-thioguanine or the 6-thioguanine hydrolysis product is greater than about 450 pmol per 8×10⁸ red blood cells. Further embodiments of these methods are described in detail below.

In another aspect, the invention provides methods for determining the amount of thiopurine drug metabolites in a sample, the methods comprising: (a) providing a sample comprising one or more 6-methylmercaptopurine nucleotide compounds; (b) converting the one or more 6-methyl-mercaptopurine nucleotide compounds to 6-methylmercaptopurine or a 6-methylmercaptopurine hydrolysis product; (c) chromatographically separating the 6-methylmercaptopurine or the 6-methylmercaptopurine hydrolysis product from other components in the converted sample using liquid chromatography; and (d) analyzing the chromatographically separated 6-methylmercaptopurine or the 6-methylmercaptopurine hydrolysis product by mass spectrometry to determine the amount of the one or more 6-methylmercaptopurine nucleotide compounds in the sample. In some embodiments, the sample originates from a biological fluid, such as whole blood. In some such embodiments, the method comprises determining a relative amount of the one or more 6-methylmercaptopurine nucleotide compounds to red blood cells in the biological sample. In some such embodiments, the amount of the one or more 6-methylmercaptopurine nucleotide compounds are correlated to assess the efficacy or toxicity of treatment of a subject undergoing treatment with a thiopurine drug. In certain embodiments, the therapeutic range for the 6-methylmercaptopurine or a 6-methylmercaptopurine hydrolysis product is less than about 5700 pmole per 8×10⁸ red blood cells. Also, in certain embodiments, the toxic value for the 6-methylmercaptopurine or a 6-methylmercaptopurine hydrolysis product is greater than about 5700 pmole per 8×10⁸ red blood cells. Further embodiments of these methods are described in detail below.

In another aspect, the invention provides systems for performing the methods described herein. For example, in certain embodiments, the invention comprises a system for determining the amount of thiopurine drug metabolites in a sample, the systems comprising: (a) a sample comprising one or more 6-thioguanine nucleotide compounds and/or one or more 6-methylmercaptopurine nucleotide compounds; (b) a station for converting the one or more 6-thioguanine nucleotide compounds to 6-thioguanine or a 6-thioguanine hydrolysis product and/or for converting the one or more 6-methyl-mercaptopurine nucleotide compounds to 6-methylmercaptopurine or a 6-methylmercaptopurine hydrolysis product; (c) a station for chromatographically separating the 6-thioguanine or the 6-thioguanine hydrolysis product from other components in the converted sample using liquid chromatography, and/or for chromatographically separating the 6-methylmercaptopurine or the 6-methylmercaptopurine hydrolysis product from other components in the converted sample using liquid chromatography; and (d) a station for analyzing the chromatographically separated 6-thioguanine or the 6-thioguanine hydrolysis product by mass spectrometry to determine the amount of the one or more 6-thioguanine nucleotide compounds in the sample, and/or for analyzing the chromatographically separated 6-methylmercaptopurine or the 6-methylmercaptopurine hydrolysis product by mass spectrometry to determine the amount of the one or more 6-methylmercaptopurine nucleotide compounds in the sample. In embodiments where the sample comprises a biological sample, the biological sample can be one or more of whole blood, red blood cells, plasma, serum, urine, cerebrospinal fluid, tissue homogenate, saliva, amniotic fluid, bile, mucus, peritoneal fluid, or lymphatic fluid. In some embodiments, the system further comprises a station for preparing the sample from a biological fluid, such as whole blood.

In certain embodiments, the system comprises a station for correlating the amounts of the one or more 6-thioguanine nucleotide compounds and the one or more 6-methylmercaptopurine nucleotide compounds to assess the efficacy or toxicity of treatment of a subject undergoing treatment with a thiopurine drug. In certain embodiments, the therapeutic range for the 6-thioguanine or the 6-thioguanine hydrolysis product is about 235-450 pmol per 8×10⁸ red blood cells. In certain embodiments, the toxic value for the 6-thioguanine or the 6-thioguanine hydrolysis product is greater than about 450 pmol per 8×10⁸ red blood cells. In certain embodiments, the therapeutic range for the 6-methylmercaptopurine or a 6-methylmercaptopurine hydrolysis product is less than about 5700 pmole per 8×10⁸ red blood cells. Also, in certain embodiments, the toxic value for the 6-methylmercaptopurine or a 6-methylmercaptopurine hydrolysis product is greater than about 5700 pmole per 8×10⁸ red blood cells.

Further aspects of the invention are described in detail below.

### BRIEF DESCRIPTION OF THE DRAWINGS

FIG. 1a shows representative chromatograms and calibration curves for the chromatographic analysis of 6-thioguanine (6-TG) and FIG lb shows representative chromatograms and calibration curves for the chromatographic analysis 4-amino-5-(methylthio) carbonyl imidazole (6-MMP*) in accordance with certain embodiments of the present invention.

FIG. 2A shows a representative separation analysis of 6-thioguanine (6-TG), 4-amino-5-(methylthio) carbonyl imidazole (6-MMP*), and 6-methylmercaptopurine (6-MMP) in accordance with certain embodiments of the present invention.

FIG. 2B shows the acid-promoted conversion of 6-thio-guanosine-5′-mono, di, and tri phosphate into 6-thioguanine as a function of time in accordance with certain embodiments of the present invention.

FIG. 2C shows the acid-promoted conversion of 6-methylmercaptopurine into 4-amino-5-(methylthio) carbonyl imidazole as a function of time in accordance with certain embodiments of the present invention.

FIG. 2D shows the complete acid-promoted conversion of 6-methylthioguanosine-5′-mono, di, and tri phosphate into 4-amino-5-(methylthio) carbonyl imidazole as a function of time in accordance with certain embodiments of the present invention.

### DETAILED DESCRIPTION

The following description recites various aspects and embodiments of the present invention. No particular embodiment is intended to define the scope of the invention. Rather, the embodiments merely provide non-limiting examples various methods and systems that are at least included within the scope of the invention. The description is to be read from the perspective of one of ordinary skill in the art; therefore, information well known to the skilled artisan is not necessarily included.

Various abbreviations may be used in the application. In most, if not all, instances, the meanings of such abbreviations are known to those of skill in the art. These abbreviations include the following abbreviations, whose meanings are provided.
APCI=atmospheric pressure chemical ionization
CBP=competitive binding protein
HTLC=high turbulence (throughput) liquid chromatography
HPLC=high performance liquid chromatography
LLE=liquid-liquid extraction
LOQ=limits of quantification
LLOQ=lower limit of quantification
ELISA=enzyme linked immunoassay
SST=system suitability test
ULOQ=upper limit of quantification
2D-LC-MS/MS=two-dimensional liquid chromatography hyphenated to tandem mass spectrometry
(LC)-LC-MS/MS=two-dimensional liquid chromatography tandem hyphenated to mass spectrometry
(LC)-MS/MS=liquid chromatography hyphenated to tandem mass spectrometry

### Definitions

The following terms, unless otherwise indicated, shall be understood to have the following meanings.

As used herein, the terms “a,” “an,” and “the” can refer to one or more unless specifically noted otherwise.

As used herein, “or” does not imply mutual exclusivity. For example, the phrase “X comprises A or B” can refer to any of the following embodiments: where X includes A and not B; where X includes B but not A; where X includes both A and B.

As used herein, the term “6-thioguanine nucleotide compound” refers to a compound that includes a 6-thioguanine unit that is covalently bound to at least a portion of a nucleotide unit, such as a phosphorylated pentose group. The term “6-methylmercaptopurine nucleotide compound” refers to a compound that includes a 6-methylmercaptopurine unit that is covalently bound to at least a portion of a nucleotide unit, such as a phosphorylated pentose group.

The term “hydrolysis product” refers to the compound obtained following an acid-catalyzed or base-catalyzed hydrolysis reaction of the precursor compound. In some embodiments, the “hydrolysis product” refers to the product obtained following an acid-catalyzed hydrolysis reaction of the precursor compound.

Throughout this application, the term “about” is used to indicate that a value includes the inherent variation of error for the device, the method being employed to determine the value, or the variation that exists among the study subjects.

As used herein, the terms “subject,” “individual,” and “patient” are used interchangeably. The use of these terms does not imply any kind of relationship to a medical professional, such as a physician.

As used herein, the phrase “liquid chromatography” or “LC” is used to refer to a process for the separation of one or more molecules or analytes in a sample from other analytes in the sample. LC involves the slowing of one or more analytes of a fluid solution as the fluid uniformly moves through a column of a finely divided substance. The slowing results from the distribution of the components of the mixture between one or more stationery phases and the mobile phase. LC includes, for example, reverse phase liquid chromatography (RPLC) and high pressure liquid chromatography (HPLC).

As used herein, the term “separate” or “purify” or the like are not used necessarily to refer to the removal of all materials other than the analyte of interest from a sample matrix. Instead, in some embodiments, the terms are used to refer to a procedure that enriches the amount of one or more analytes of interest relative to one or more other components present in the sample matrix. In some embodiments, a “separation” or “purification” may be used to remove or decrease the amount of one or more components from a sample that could interfere with the detection of the analyte, for example, by mass spectrometry.

As used herein, the term “mass spectrometry” or “MS” analysis refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules may be ionized and detected by any suitable means known to one of skill in the art. The phrase “tandem mass spectrometry” or “MS/MS” is used herein to refer to a technique for the identification and/or quantitation of molecules in a sample, wherein multiple rounds of mass spectrometry occur, either simultaneously using more than one mass analyzer or sequentially using a single mass analyzer. As used herein, a “mass spectrometer” is an apparatus that includes a means for ionizing molecules and detecting charged molecules.

As used herein, “electrospray ionization” or “ESI” refers to a technique used in mass spectrometry to ionize molecules in a sample while avoiding fragmentation of the molecules. The sample is dispersed by the electrospray into a fine aerosol. The sample will typically be mixed with a solvent, usually a volatile organic compound (e.g., methanol or acetonitrile) mixed with water. The aerosol is then transferred to the mass spectrometer through a capillary, which can be heated to aid further solvent evaporation from the charged droplets.

As used herein, a “quadrupole analyzer” is a type of mass analyzer used in MS. It consists of four circular rods (two pairs) that are set highly parallel to each other. The quadrupole analyzer is the component of the instrument that organizes the charged particles of the sample based on their mass-to-charge ratio. One of skill in the art would understand that use of a quadrupole analyzer can lead to increased specificity of results. One pair of rods is set at a positive electrical potential and the other set of rods is at a negative potential. To be detected, an ion must pass through the center of a trajectory path bordered and parallel to the aligned rods. When the quadrupoles are operated at a given amplitude of direct current and radio frequency voltages, only ions of a given mass-to-charge ratio will resonate and have a stable trajectory to pass through the quadrupole and be detected. As used herein, “positive ion mode” refers to a mode wherein positively charged ions are detected by the mass analyzer, and “negative ion mode” refers to a mode wherein negatively charged ions are detected by the mass analyzer. For “selected ion monitoring” or “SIM,” the amplitude of the direct current and the radio frequency voltages are set to observe only a specific mass.

As used herein, the term “analytical column” refers to a chromatography column having sufficient chromatographic plates to effect a separation of the components of a test sample matrix. Preferably, the components eluted from the analytical column are separated in such a way to allow the presence or amount of an analyte(s) of interest to be determined. In some embodiments, the analytical column comprises particles having an average diameter of about 5 μm. In some embodiments, the analytical column is a functionalized silica or polymer-silica hybrid, or a polymeric particle or monolithic silica stationary phase, such as a phenyl-hexyl functionalized analytical column.

As used herein, the term “hemolysed” refers to the rupturing of the red blood cell membrane, which results in the release of hemoglobin and other cellular contents into the plasma or serum and the term “lipemic” refers to an excess of fats or lipids in blood. As used herein, the term “thiopurine treatment” refers to a treatment regimen that includes administration of a thiopurine drug to a subject (e.g., a human subject). The term “thiopurine drug” refers to various purine antimetabolites, including but not limited to 6-thioguanine, 6-mercaptopurine, and azathiopurine. Such drugs can be used for the treatment of various conditions, including but not limited to autoimmune disorders, such as Crohn's disease and irritable bowel syndrome (IBS).

### Determining Amount of 6-Thioguanine (6-TG) Nucleotide Compounds in a Sample

In another aspect, the invention provides methods for determining the amount of thiopurine drug metabolites in a sample, the methods comprising: (a) providing a sample comprising one or more 6-thioguanine nucleotide compounds; (b) converting the one or more 6-thioguanine nucleotide compounds to 6-thioguanine or a 6-thioguanine hydrolysis product; (c) chromatographically separating the 6-thioguanine or the 6-thioguanine hydrolysis product from other components in the converted sample using liquid chromatography; (d) analyzing the chromatographically separated 6-thioguanine or the 6-thioguanine hydrolysis product by mass spectrometry to determine the amount of the one or more 6-thioguanine nucleotide compounds in the sample.

These methods comprise providing a sample containing one or more 6-thioguanine nucleotide compounds. In this context, the term “providing” is to be construed broadly. The term is not intended to refer exclusively to a subject who provided a biological sample. For example, a technician in an off-site clinical laboratory can be said to “provide” the sample, for example, as the sample is prepared for purification by chromatography.

The sample is not limited to any particular sample type. The sample contains one or more 6-thioguanine nucleotide compounds, but, in general, also includes other components. In some embodiments, the sample is a sample that has been processed and prepared for reduction and/or hydrolysis and/or purification by chromatography. Such processing may be useful for optimizing the effectiveness of subsequent purification steps. Such processing methods are well known to those of skill in the art.

The invention is not limited to any particular means of sample handling. In some embodiments, it may be useful to separate the sample into two or more fractions prior to purification by extraction and/or chromatography. In some such embodiments, two or more of such fractions may be prepared differently, for example, to help improve the sensitivity or selectivity of the separation for a particular column chemistry. In some embodiments, the method includes preparing a single sample for repeat injections across multiple liquid chromatography systems.

The invention is not limited to any particular sample size. In some embodiments, the sample comprises a biological sample. In such embodiments, the sample may also include other components, such as solvents, buffers, anticlotting agents and the like. In embodiments where the sample comprises a biological sample, the biological sample can be one or more of whole blood, red blood cells, plasma, serum, urine, cerebrospinal fluid, tissue homogenate, saliva, amniotic fluid, bile, mucus, peritoneal fluid, or lymphatic fluid. The invention is not limited to any particular volume of biological sample. In some embodiments, the biological sample is at least about 50-1000 μL, at least about 100-800 μL, at least about 200-700 μL, or at least about 400-600 μL in volume. In certain embodiments, the biological sample is at least about 500 μL in volume.

The method comprises converting the one or more 6-thioguanine nucleotide compounds to 6-thioguanine or a 6-thioguanine hydrolysis product. The converting can be carried out in any manner known to those of skill in the art. In some embodiments, the converting comprises contacting the one or more 6-thioguanine nucleotide compounds with a sulfur-containing reducing agent to yield one or more reduced 6-thioguanine nucleotide compounds. Any suitable sulfur-containing reducing agent can be used. In some embodiments, the sulfur-containing reducing agent is dithioerythritol. In some such embodiments, the method comprises contacting the one or more reduced 6-thioguanine nucleotide compounds with an acidic hydrolyzing agent. Any suitable acidic hydrolyzing agent can be used. In some embodiments, the acidic hydrolyzing agent is perchloric acid, nitric acid, sulfuric acid, or a combination thereof. In some such embodiments, the acidic hydrolyzing agent is perchloric acid.

In some embodiments the hydrolysis of 6-thioguanine nucleotide compounds proceeds according to Scheme 1 below. 6-Thioguanine nucleotides may be present in the sample as a mixture of 6-thioguanine nucletotides with differing amounts of phosphorylation. In one embodiment, 6-thioguanine nucleotides are present as a mixture of species having one, two, or three phosphate groups attached to the ribose portion of the 6-thioguanine nuecleotide. In some embodiments, acid hydrolysis of the mixture of 6-thioguanine nucleotides produces 6-thioguanine, with the ribose and phosphate fragments removed. Thus, the hydrolysis of the mixture of 6-thioguanine nucleotides provides the total amount of 6-thioguanine in the sample.

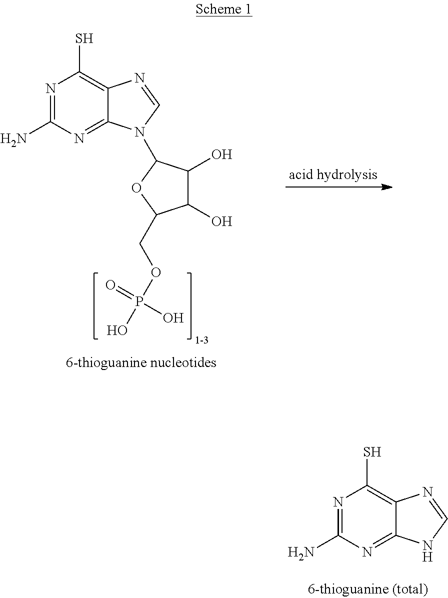

In some embodiments the hydrolysis of 6-methylmercaptopurine nucleotide compounds proceeds according to Scheme 2 below. 6-Methylmercaptopurine nucleotides may be present in the sample as a mixture of 6-methylmercaptopurine nucletotides with differing amounts of phosphorylation. In one embodiment, 6-methylmercaptopurine nucleotides are present as a mixture of species having one, two, or three phosphate groups attached to the ribose portion of the 6-methylmercaptopurine. In some embodiments, acid hydrolysis of the mixture of 6-methylmercaptopurine nucleotides produces 6-methylmercaptopurine as an acid-unstable intermediate, with the ribose and phosphate fragments removed. The 6-methylmercaptopurine is readily hydrolyzed in the presence of acid to form a 6-methylmercaptopurine hydrolysis product comprising 4-amino-5-(methylthio) carbonyl imidazole. In one embodiment, the 6-methylmercaptopurine hydrolysis product is 4-amino-5-(methylthio) carbonyl imidazole. Thus, the hydrolysis of the mixture of 6-thioguanine nucleotides and 6-methylmercaptopurine nucleotides provides the total amount of 6-methylmercaptopurine in the sample as measured by the amount of 4-amino-5-(methylthio) carbonyl imidazole after acid hydrolysis.

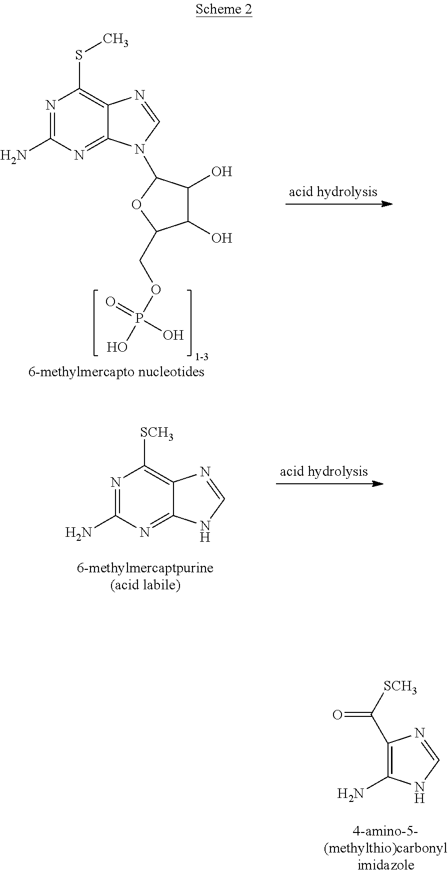

The hydrolysis can be carried out for any suitable time and at any suitable temperature. In some embodiments, the hydrolysis is carried out for a time ranging from 30 minutes to 4 hours, or from 1 hour to 3 hours. In some embodiments, the hydrolysis is carried out for about 1 hour, or about 2 hours, or about 3 hours. In some embodiments, the temperature is no more than the boiling point of the solution containing the analyte, and is at least 60° C., or at least 70° C., or at least 80° C., or at least 90° C.

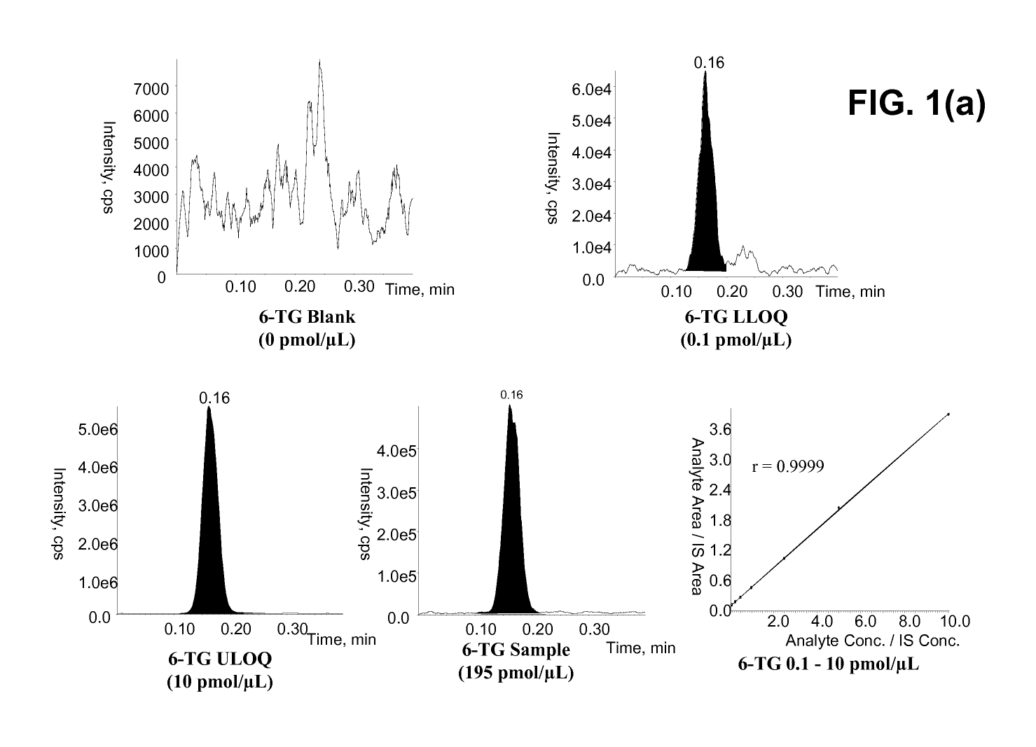

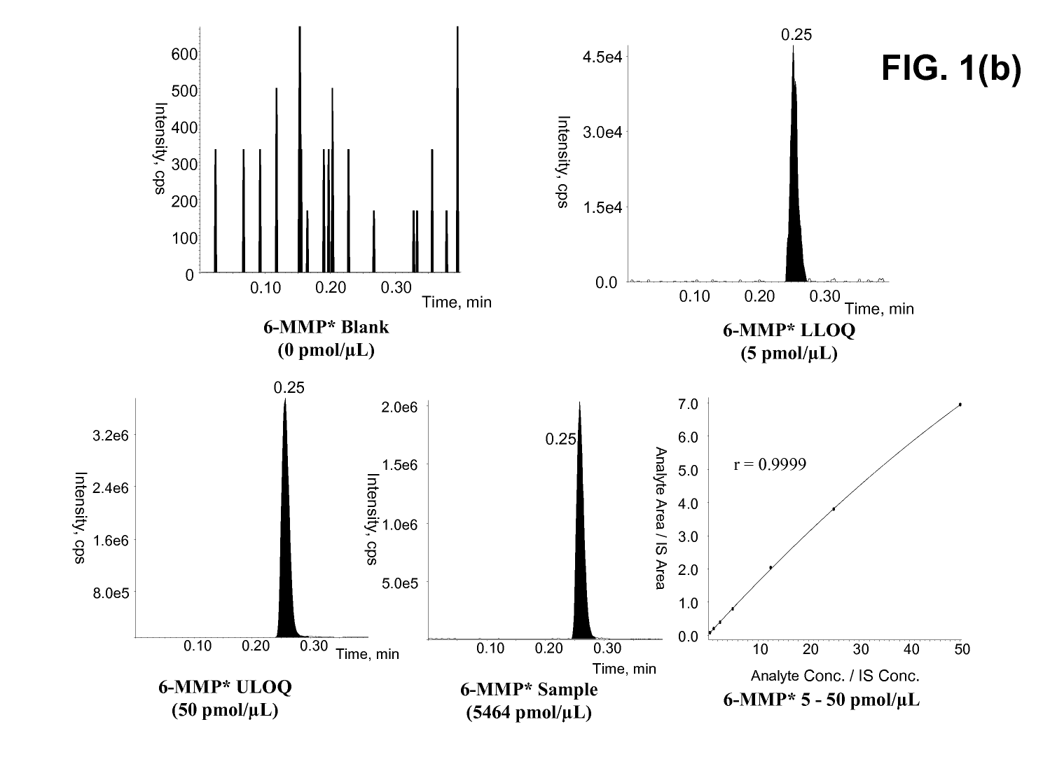

FIG. 1 shows representative calibration curves for the chromatographic analysis of a) 6-thioguanine (6-TG) and b) 4-amino-5-(methylthio) carbonyl imidazole (6-MMP*). In a non-limiting example, the chromatograms in FIG. 1 were obtained on an Aria Transcend TX4 chromatograph with an injection volume of about 20-30 μL. In a non-limiting example, the analytical column comprised an Atlantis T3, 2.1×50 mm, 3 μm coumn. In one embodiment, the mobile phases comprised of a first mobile phase (Mobile Phase A), comprising about 0.1% aqueous formic acid, and a second mobile phase (Mobile Phase B), comprising 0.1% formic acid in acetonitrile. In some embodiments, the flow rate was between 1.0 and 2.0 mL/min. In one embodiment, the flow rate was about 1.25 mL/min. In one embodiment, the elution (separation) protocol comprised about 0-5 sec 0% B, about a 5-35 sec ramp to 30% B, about 35-65 sec to 100% B, about 65-85 sec hold at 100% B, and about 85-130 sec step to 0% B. Thus, it can be seen that at least in certain embodiments, for detection of 6-thioguanine (6-TG) or the 6-thioguanine hydrolysis product, the lower limit of quantitation (LLOQ) is about 0.1 pmol/μL and the upper limit of quantitation (ULOQ) is about 10 pmol/μL, with a linear correlation coefficient of r=0.9999 between the LLOQ (FIG. 1a). Also, in at least certain embodiments, for detection of 6-methylmercaptopurine or the 6-methylmercaptopurine hydrolysis product the lower limit of quantitation (LLOQ) is about 5 pmol/μL and the upper limit of quantitation (ULOQ) is about 50 pmol/μL, with a linear correlation coefficient of r=0.9999 between the LLOQ (FIG. 1b).

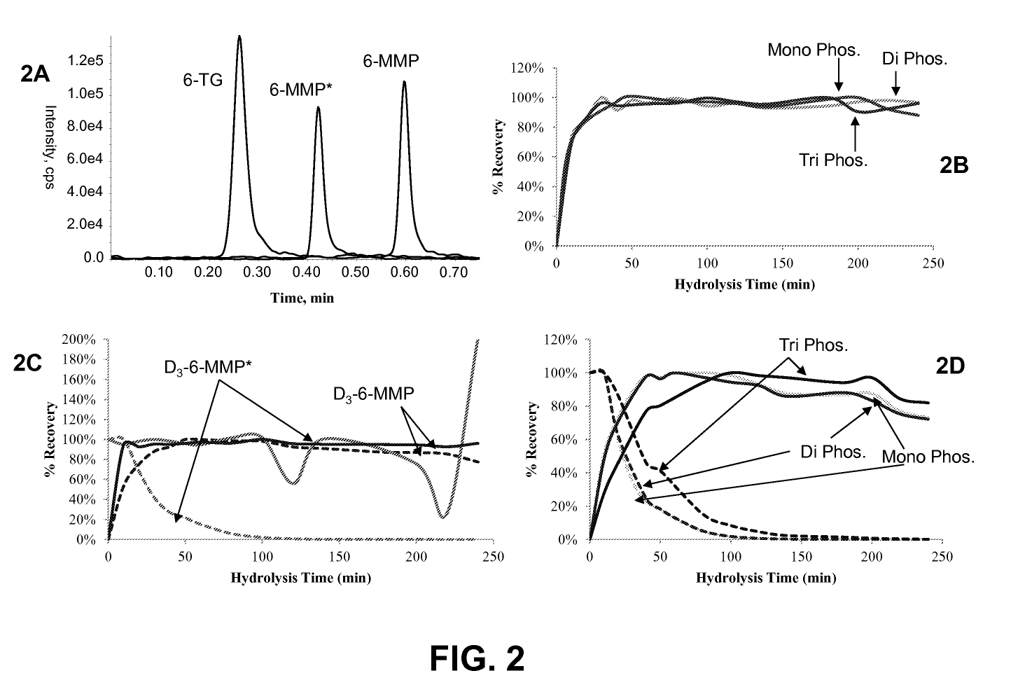

FIG. 2A shows a representative separation analysis of 6-thioguanine (6-TG), 4-amino-5-(methylthio) carbonyl imidazole (6-MMP*), and 6-methylmercaptopurine (6-MMP). Each thioguanine species or derivative thereof was readily resolved and separable from each other.

FIG. 2B shows the acid-promoted conversion of 6-thio-guanosine-5′-mono, di, and tri phosphate into 6-thioguanine as a function of time. Each line in the graph in FIG. 2B represents a different phosphorylation state (i.e. mono, di, or tri). Generally it was observed that the conversion into 6-thioguanine did not have a strong dependence on the number of phosphate groups. In some embodiments, the complete conversion of 6-thio-guanosine-5′-mono, di, and tri phosphate into 6-thioguanine occurs in less than 60 minutes. In some embodiments, the conversion occurs in less than 50 minutes. In some embodiments, the conversion occurs in about 45 minutes.

FIG. 2C shows the acid-promoted conversion of 6-methylmercaptopurine into 4-amino-5-(methylthio) carbonyl imidazole (6-MMP*) as a function of time. Generally, the conversion of 6-MMP to 6-MMP* occurred in less than 140 minutes. In some embodiments, the conversion occurs in about 120 minutes. In some embodiments, the conversion occurs in about 100 minutes. The conversion of 6-MMP to 6-MMP* is represented in FIG. 2C by the dashed lines. An internal deuterated standard, D₃-6-MMP converted to D₃-6-MMP* at the same rate as unlabeled 6-MMP, allowing the concentration of 6-MMP (and thus 6-MMP nucleotides) to be determined by the amount of 6-MMP* detected. The deuterium was incorporated into D₃-6-MMP in the d₃-methyl group attached to the sulphur in D₃-6-MMP. The conversion of 6-MMP nucleotides into 6-MMP* is represented by the solid lines in FIG. 2C.

FIG. 2D shows the complete acid-promoted conversion of 6-methylthioguanosine-5′-mono, di, and tri phosphate into 4-amino-5-(methylthio) carbonyl imidazole as a function of time.

The converting step results in a sample comprising 6-thioguanine or a 6-thioguanine hydrolysis product. In some embodiments, the converted sample comprises 6-thioguanine and substantially no 6-thioguanine hydrolysis product (i.e., less than 10 ppm, or less than 1 ppm). In some other embodiments, the converted sample comprises 6-thioguanine hydrolysis product and substantially no 6-thioguanine (i.e., less than 10 ppm, or less than 1 ppm). In some other embodiments, the converted sample comprises both 6-thioguanine and a 6-thioguanine hydrolysis product. In some such embodiments, the molar ratio of the 6-thioguanine hydrolysis product to 6-thioguanine is at least 2:1, or at least 3:1, or at least 4:1, or at least 5:1, or at least 10:1, or at least 25:1, or at least 100:1. The 6-thioguanine hydrolysis product can be any compound or plurality of compounds formed upon acid-catalyzed hydrolysis of 6-thioguanine In some embodiments, the sample contains both phosphorylated (i.e. nucleotide) and un-phosphorylated forms of 6-thioguanine. In some embodiments, the total amount of 6-thioguanine hydrolysis product comprises amounts derived from conversion of both 6-thioguanine (i.e un-phosphorylated) and 6-thioguanine nucleotide.

The converting steps described above can be carried out in any suitable fashion according to the knowledge of those of skill in the art. In some embodiments, the converting steps are carried out manually. In other embodiments, the converting steps are carried out in an automated manner. Further, in some embodiments of the invention, the converted sample can undergo one or more processing steps before chromatographic separation, including buffering and the like.

The methods comprise chromatographically separating 6-thioguanine or a 6-thioguanine hydrolysis product using liquid chromatography. In some embodiments, the focus of the chromatographic separation is only on one of the 6-thioguanine or the 6-thioguanine hydrolysis product. In general, the converted sample should contain sufficient quantities of the desired analyte so as to allow the separation to be effective. The invention is not limited to any particular manner of performing liquid chromatography. In general, the chromatographic separation step includes using at least one liquid chromatography (LC) column. In some embodiments, multiple LC columns are used, such as two or more, or three or more, or four or more LC columns. In some such embodiments two, three, four, five, six, eight, or ten LC columns are used. In some such embodiments, two or more of these LC columns are arranged parallel to each other, and are connected inline to the same mass spectrometer.

The invention is not limited to any particular types of columns. Any column suitable for the separation of 6-thioguanine or a 6-thioguanine hydrolysis product can be used. In some embodiments, one or more analytical columns are used. In some such embodiments, one or more reverse phase columns are used. In some embodiments, the method employs two or more reverse phase columns in parallel, which are connected inline to the same mass spectrometer. In some embodiments, a HILIC column is used.

Further, the invention is not limited to any particular mobile phase. Any suitable mobile phase can be used, as long as the mobile phase is suitable for use with a particular LC column and for chromatographically separating 6-thioguanine or a 6-thioguanine hydrolysis product in the LC column. In some embodiments, the mobile phase is a polar solvent system. The polar solvent system can include one or more polar solvents, including but not limited to water, methanol, acetonitrile, or a mixture of two or more of the foregoing. In some such embodiments, the mobile phase employs a gradient, such that the relative ratios of two or more solvents are varied over time.

As noted above, two or more LC columns (e.g., reverse phase columns) can be used in parallel and connected inline to the same mass spectrometer, e.g., to improve throughput. In some such embodiments, the sample is introduced to the two or more LC columns at different times. In some embodiments, the introduction of the test sample to the two or more LC columns is staggered, meaning that there is a pre-determined time interval separating the introduction of sample to two or more LC columns. Appropriate time intervals can be selected based on various factors, including the elution time, column chemistries, and the potential need to avoid interfering with the analysis of 6-thioguanine or a 6-thioguanine hydrolysis product eluted from one or more of the other LC columns.

In some embodiments of the invention, one or more additional LC columns can be placed in series with another column. In some embodiments, a guard column is placed in parallel with another LC column, and both the guard column and the LC column are reverse phase columns. Such series of two or more columns can also be arranged in parallel, such that there are two or more series of columns operating in parallel, where each series contains two or more columns.

The methods comprise analyzing the chromatographically separated 6-thioguanine or a 6-thioguanine hydrolysis product by mass spectrometry to determine the amount of the 6-thioguanine nucleotide compound in the sample. In some embodiments, two or more of the LC columns feed into the same mass spectrometer. In some further embodiments, three or more of the LC columns feed into the same mass spectrometer. In some embodiments, the mass spectrometer is part of a combined LC-MS system.

The invention is not limited to any particular type of mass spectrometer. Any suitable mass spectrometer can be used. In some embodiments, the method employs a tandem mass spectrometer. In some such embodiments, analyzing the 6-thioguanine or a 6-thioguanine hydrolysis product can include, ionizing the compound, analyzing the ionized the compound, fragmenting the compound ion into two or more fragment ions, and analyzing the fragment ions. The invention is not limited to a mass spectrometer using any particular ionization methods. Any suitable ionization can be used. Suitable ionization methods include, but are not limited to photoionization, electrospray ionization, atmospheric pressure ionization, and electron capture ionization. And in embodiments that employ fragmenting, any suitable fragmentation technique can be used. Suitable techniques include, but are not limited to collision induced dissociation, electron capture dissociation, electron transfer dissociation, infrared multiphoton dissociation, radiative dissociation, electron-detachment dissociation, and surface-induced dissociation.

In some embodiments, the tandem mass spectrometer is a MDS-Sciex API5000 triple quadrupole mass spectrometer. In some embodiments, the tandem mass spectrometer has an atmospheric pressure ionization source, and the analyzing step comprises an ionization method selected from the group consisting of photoionization, electrospray ionization (ESI), atmospheric pressure chemical ionization (APCI), electron capture ionization, electron ionization, fast atom bombardment/liquid secondary ionization (FAB/LSI), matrix assisted laser desorption ionization (MALDI), field ionization, field desorption, thermospray/plasmaspray ionization, and particle beam ionization. The ionization method may be in positive ion mode or negative ion mode. The analyzing step may also include multiple reaction monitoring or selected ion monitoring (SIM), and the two or more biomolecules are analyzed simultaneously or sequentially. In some embodiments, the analyzing step uses a quadrupole analyzer. In some embodiments, the mass spectrometer is a triple quadrupole mass spectrometer.

In some embodiments, the methods employ one or more internal standards. In some such embodiments, the internal standards are introduced into the sample prior to the converting step. Any suitable internal standards can be used. In some embodiments, at least one of the internal standards is selected from stable isotopically-labeled forms of 6-thioguanine or the 6-thioguanine hydrolysis product. In some such embodiments the internal standard comprises an amount of a stable isotopically-labeled form of 6-thioguanine. Such internal standards can employ any suitable stable isotopes of atoms included in the compound, including but not limited to deuterium, carbon-13, nitrogen-15, sulfur-34, or any combination thereof.

The methods also include determining the amount of the one or more 6-thioguanine nucleotide compounds in the sample, for example, based on the results of the analysis by mass spectrometry. The invention is not limited to any particular technique for conducting this analysis. Any suitable technique known in the art can be used. In some embodiments, this includes correlating the detected amounts of one or more internal standards against certain reference values to determine the amount of the one or more 6-thioguanine nucleotide compounds in the original sample.

The invention is not limited to any particular source of the sample. In some embodiments, however, the sample is derived from a biological sample, which is taken from a mammalian subject, such as a human subject. The invention is not limited to any particular biological sample. In some embodiments, the biological sample is whole blood, red blood cells, plasma, serum, urine, cerebrospinal fluid, tissue homogenate, saliva, amniotic fluid, bile, mucus, peritoneal fluid, lymphatic fluid, or a combination thereof. In some embodiments, the biological sample is whole blood. Techniques for handling biological samples and preparing such samples for quantitative and/or qualitative analysis are well known in the art.

In some embodiments, the methods can include determining a relative amount of the one or more 6-thioguanine nucleotide compounds to red blood cells in the biological sample. This can be done using techniques known in the art. In some embodiments, the value obtained from this analysis can be used to assess certain features related to the efficacy or toxicity of a thiopurine treatment in a subject, such as a human subject.

In some embodiments, the methods include correlating the relative amount of the one or more 6-thioguanine nucleotide compounds in the biological sample to the efficacy of thiopurine treatment of a human subject. In some embodiments, such efficacy can be identified where the relative amount of the one or more 6-thioguanine nucleotide compounds to red blood cells in the biological sample ranges from 100 to 450 pmol per 8×10⁸ red blood cells, or from 200 to 450 pmol per 8×10⁸ red blood cells, or from 235 to 450 pmol per 8×10⁸ red blood cells. In some other embodiments, the methods include correlating the relative amount of the one or more 6-thioguanine nucleotide compounds in the biological sample to an increased risk of leucopenia. In some embodiments, such increased risk can be identified where the relative amount of the one or more 6-thioguanine nucleotide compounds to red blood cells in the biological sample is at least 450 pmol per 8×10⁸ red blood cells, or at least 500 pmol per 8×10⁸ red blood cells.

In some embodiments, the methods can include generating a report that recites the concentration of 6-thioguanine (for example, as the one or more 6-thioguanine nucleotide compounds) in the biological sample. In some such embodiments, such reports are provided to treating physicians or other health-care professionals for use in monitoring or modifying a subject's thiopurine treatment. Such reports can also include suggestions regarding efficacy or toxicity related to the thiopurine treatment.

### Determining Amount of 6-Methylmercaptopurine (6-MMP) Nucleotide Compounds in a Sample

In another aspect, the invention provides methods for determining the amount of thiopurine drug metabolites in a sample, the methods comprising: (a) providing a sample comprising one or more 6-methylmercaptopurine nucleotide compounds; (b) converting the one or more 6-methylmercaptopurine nucleotide compounds to 6-methylmercaptopurine or a 6-methylmercaptopurine hydrolysis product; (c) chromatographically separating the 6-methylmercaptopurine or the 6-methylmercaptopurine hydrolysis product from other components in the converted sample using liquid chromatography; (d) analyzing the chromatographically separated 6-methylmercaptopurine or the 6-methylmercaptopurine hydrolysis product by mass spectrometry to determine the amount of the one or more 6-methylmercaptopurine nucleotide compounds in the sample.

These methods comprise providing a sample containing one or more 6-methylmercaptopurine nucleotide compounds. In this context, the term “providing” is to be construed broadly. The term is not intended to refer exclusively to a subject who provided a biological sample. For example, a technician in an off-site clinical laboratory can be said to “provide” the sample, for example, as the sample is prepared for purification by chromatography.

The sample is not limited to any particular sample type. The sample contains one or more 6-methylmercaptopurine nucleotide compounds, but, in general, also includes other components. In some embodiments, the sample is a sample that has been processed and prepared for reduction and/or hydrolysis and/or purification by chromatography. Such processing may be useful for optimizing the effectiveness of subsequent purification steps. Such processing methods are well known to those of skill in the art.

The invention is not limited to any particular means of sample handling. In some embodiments, it may be useful to separate the sample into two or more fractions prior to purification by extraction and/or chromatography. In some such embodiments, two or more of such fractions may be prepared differently, for example, to help improve the sensitivity or selectivity of the separation for a particular column chemistry. In some embodiments, the method includes preparing a single sample for repeat injections across multiple liquid chromatography systems.

The invention is not limited to any particular sample size. In some embodiments, the sample comprises a biological sample. In such embodiments, the sample may also include other components, such as solvents, buffers, anticlotting agents and the like. In embodiments where the sample comprises a biological sample, the biological sample can be one or more of whole blood, red blood cells, plasma, serum, urine, cerebrospinal fluid, tissue homogenate, saliva, amniotic fluid, bile, mucus, peritoneal fluid, or lymphatic fluid. The invention is not limited to any particular volume of biological sample. In some embodiments, the biological sample is at least about 50-1000 μL, at least about 100-800 μL, at least about 200-700 μL, or at least about 400-600 μL in volume. In certain embodiments, the biological sample is at least about 500 μL in volume.

The method comprises converting the one or more 6-methylmercaptopurine nucleotide compounds to 6-methylmercaptopurine or a 6-methylmercaptopurine hydrolysis product. The converting can be carried out in any manner known to those of skill in the art. In some embodiments, the converting comprises contacting the one or more 6-methylmercaptopurine nucleotide compounds with a sulfur-containing reducing agent to yield one or more reduced 6-methylmercaptopurine nucleotide compounds. Any suitable sulfur-containing reducing agent can be used. In some embodiments, the sulfur-containing reducing agent is dithioerythritol. In some such embodiments, the method comprises contacting the one or more reduced 6-methylmercaptopurine nucleotide compounds with an acidic hydrolyzing agent. Any suitable acidic hydrolyzing agent can be used. In some embodiments, the acidic hydrolyzing agent is perchloric acid, nitric acid, sulfuric acid, or a combination thereof. In some such embodiments, the acidic hydrolyzing agent is perchloric acid.

The hydrolysis can be carried out for any suitable time and at any suitable temperature. In some embodiments, the hydrolysis is carried out for a time ranging from 30 minutes to 4 hours, or from 1 hour to 3 hours. In some embodiments, the hydrolysis is carried out for about 1 hour, or about 2 hours, or about 3 hours. In some embodiments, the temperature is no more than the boiling point of the solution containing the analyte, and is at least 60° C., or at least 70° C., or at least 80° C., or at least 90° C.

The converting step results in a sample comprising 6-methylmercaptopurine or a 6-methylmercaptopurine hydrolysis product. In some embodiments, the converted sample comprises 6-methylmercaptopurine and substantially no 6-methylmercaptopurine hydrolysis product (i.e., less than 10 ppm, or less than 1 ppm). In some other embodiments, the converted sample comprises 6-methylmercaptopurine hydrolysis product and substantially no 6-methylmercaptopurine (i.e., less than 10 ppm, or less than 1 ppm). In some other embodiments, the converted sample comprises both 6-methylmercaptopurine and a 6-methylmercaptopurine hydrolysis product. In some such embodiments, the molar ratio of the 6-methylmercaptopurine hydrolysis product to 6-methylmercaptopurine is at least 2:1, or at least 3:1, or at least 4:1, or at least 5:1, or at least 10:1, or at least 25:1, or at least 100:1. In some embodiments, the sample contains both phosphorylated (i.e. nucleotide) and un-phosphorylated forms of 6-methylmercaptopurine. In some embodiments, the total amount of 6-methylmercaptopurine hydrolysis product comprises amounts derived from conversion of both 6-methylmercaptopurine (i.e un-phosphorylated) and 6-methylmercaptopurine nucleotide.

The 6-methylmercaptopurine hydrolysis product can be any compound or plurality of compounds formed upon acid-catalyzed hydrolysis of 6-methylmercaptopurine. In some embodiments, the 6-methylmercaptopurine hydrolysis product is 4-amino-5-(methylthio)-carbonyl imidazole.

The converting steps described above can be carried out in any suitable fashion according to the knowledge of those of skill in the art. In some embodiments, the converting steps are carried out manually. In other embodiments, the converting steps are carried out in an automated manner. Further, in some embodiments of the invention, the converted sample can undergo one or more processing steps before chromatographic separation, including buffering and the like.

The methods comprise chromatographically separating 6-methylmercaptopurine or a 6-methylmercaptopurine hydrolysis product using liquid chromatography. In some embodiments, the focus of the chromatographic separation is only on one of the 6-methylmercaptopurine or the 6-methylmercaptopurine hydrolysis product. In general, the converted sample should contain sufficient quantities of the desired analyte so as to allow the separation to be effective. The invention is not limited to any particular manner of performing liquid chromatography. In general, the chromatographic separation step includes using at least one liquid chromatography (LC) column. In some embodiments, multiple LC columns are used, such as two or more, or three or more, or four or more LC columns. In some such embodiments two, three, four, five, six, eight, or ten LC columns are used. In some such embodiments, two or more of these LC columns are arranged parallel to each other, and are connected inline to the same mass spectrometer.

The invention is not limited to any particular types of columns. Any column suitable for the separation of 6-methylmercaptopurine or a 6-methylmercaptopurine hydrolysis product can be used. In some embodiments, one or more analytical columns are used. In some such embodiments, one or more reverse phase columns are used. In some embodiments, the method employs two or more reverse phase columns in parallel, which are connected inline to the same mass spectrometer. In some embodiments, a HILIC column is used.

Further, the invention is not limited to any particular mobile phase. Any suitable mobile phase can be used, as long as the mobile phase is suitable for use with a particular LC column and for chromatographically separating 6-methylmercaptopurine or a 6-methylmercaptopurine hydrolysis product in the LC column. In some embodiments, the mobile phase is a polar solvent system. The polar solvent system can include one or more polar solvents, including but not limited to water, methanol, acetonitrile, or a mixture of two or more of the foregoing. In some such embodiments, the mobile phase employs a gradient, such that the relative ratios of two or more solvents are varied over time. As noted above, two or more LC columns (e.g., reverse phase columns) can be used in parallel and connected inline to the same mass spectrometer, e.g., to improve throughput. In some such embodiments, the sample is introduced to the two or more LC columns at different times. In some embodiments, the introduction of the test sample to the two or more LC columns is staggered, meaning that there is a pre-determined time interval separating the introduction of sample to two or more LC columns. Appropriate time intervals can be selected based on various factors, including the elution time, column chemistries, and the potential need to avoid interfering with the analysis of 6-methylmercaptopurine or a 6-methylmercaptopurine hydrolysis product eluted from one or more of the other LC columns.

In some embodiments of the invention, one or more additional LC columns can be placed in series with another column. In some embodiments, a guard column is placed in parallel with another LC column, and both the guard column and the LC column are reverse phase columns. Such series of two or more columns can also be arranged in parallel, such that there are two or more series of columns operating in parallel, where each series contains two or more columns.

The methods comprise analyzing the chromatographically separated 6-methylmercaptopurine or a 6-methylmercaptopurine hydrolysis product by mass spectrometry to determine the amount of the 6-methylmercaptopurine nucleotide compound in the sample. In some embodiments, two or more of the LC columns feed into the same mass spectrometer. In some further embodiments, three or more of the LC columns feed into the same mass spectrometer. In some embodiments, the mass spectrometer is part of a combined LC-MS system.

The invention is not limited to any particular type of mass spectrometer. Any suitable mass spectrometer can be used. In some embodiments, the method employs a tandem mass spectrometer. In some such embodiments, analyzing the 6-methylmercaptopurine or a 6-methylmercaptopurine hydrolysis product can include, ionizing the compound, analyzing the ionized the compound, fragmenting the compound ion into two or more fragment ions, and analyzing the fragment ions. The invention is not limited to a mass spectrometer using any particular ionization methods. Any suitable ionization can be used. Suitable ionization methods include, but are not limited to photoionization, electrospray ionization, atmospheric pressure ionization, and electron capture ionization. And in embodiments that employ fragmenting, any suitable fragmentation technique can be used. Suitable techniques include, but are not limited to collision induced dissociation, electron capture dissociation, electron transfer dissociation, infrared multiphoton dissociation, radiative dissociation, electron-detachment dissociation, and surface-induced dissociation.

In some embodiments, the tandem mass spectrometer is a MDS-Sciex API5000 triple quadrupole mass spectrometer. In some embodiments, the tandem mass spectrometer has an atmospheric pressure ionization source, and the analyzing step comprises an ionization method selected from the group consisting of photoionization, electrospray ionization (ESI), atmospheric pressure chemical ionization (APCI), electron capture ionization, electron ionization, fast atom bombardment/liquid secondary ionization (FAB/LSI), matrix assisted laser desorption ionization (MALDI), field ionization, field desorption, thermospray/plasmaspray ionization, and particle beam ionization. The ionization method may be in positive ion mode or negative ion mode. The analyzing step may also include multiple reaction monitoring or selected ion monitoring (SIM), and the two or more biomolecules are analyzed simultaneously or sequentially. In some embodiments, the analyzing step uses a quadrupole analyzer. In some embodiments, the mass spectrometer is a triple quadrupole mass spectrometer.

In some embodiments, the methods employ one or more internal standards. In some such embodiments, the internal standards are introduced into the sample prior to the converting step. Any suitable internal standards can be used. In some embodiments, at least one of the internal standards is selected from stable isotopically-labeled forms of 6-methylmercaptopurine or the 6-methylmercaptopurine hydrolysis product. In some such embodiments the internal standard comprises an amount of a stable isotopically-labeled form of 6-methylmercaptopurine. Such internal standards can employ any suitable stable isotopes of atoms included in the compound, including but not limited to deuterium, carbon-13, nitrogen-15, sulfur-34, or any combination thereof.

The methods also include determining the amount of the one or more 6-methylmercaptopurine nucleotide compounds in the sample, for example, based on the results of the analysis by mass spectrometry. The invention is not limited to any particular technique for conducting this analysis. Any suitable technique known in the art can be used. In some embodiments, this includes correlating the detected amounts of one or more internal standards against certain reference values to determine the amount of the one or more 6-methylmercaptopurine nucleotide compounds in the original sample.

The invention is not limited to any particular source of the sample. In some embodiments, however, the sample is derived from a biological sample, which is taken from a mammalian subject, such as a human subject. The invention is not limited to any particular biological sample. In some embodiments, the biological sample is whole blood, red blood cells, plasma, serum, urine, cerebrospinal fluid, tissue homogenate, saliva, amniotic fluid, bile, mucus, peritoneal fluid, lymphatic fluid, or a combination thereof. In some embodiments, the biological sample is whole blood. Techniques for handling biological samples and preparing such samples for quantitative and/or qualitative analysis are well known in the art.

In some embodiments, the methods can include determining a relative amount of the one or more 6-methylmercaptopurine nucleotide compounds to red blood cells in the biological sample. This can be done using techniques known in the art. In some embodiments, the value obtained from this analysis can be used to assess certain features related to the efficacy or toxicity of a thiopurine treatment in a subject, such as a human subject.

In some embodiments, the methods include correlating the relative amount of the one or more 6-methylmercaptopurine nucleotide compounds in the biological sample to the efficacy of thiopurine treatment of a human subject. In some embodiments, the methods include correlating the relative amount of the one or more 6-methylmercaptopurine nucleotide compounds in the biological sample to an increased risk of hepatotoxicity. In some embodiments, such increased risk can be identified where the relative amount of the one or more 6-methylmercaptopurine nucleotide compounds to red blood cells in the biological sample is at least 5500 pmol per 8×10⁸ red blood cells, or at least 5700 pmol per 8×10⁸ red blood cells, or at least 6000 pmol per 8×10⁸ blood cells.

In some embodiments, the methods can include generating a report that recites the concentration of 6-methylmercaptopurine (for example, as the one or more 6-methylmercaptopurine nucleotide compounds) in the biological sample. In some such embodiments, such reports are provided to treating physicians or other health-care professionals for use in monitoring or modifying a subject's thiopurine treatment. Such reports can also include suggestions regarding efficacy or toxicity related to the thiopurine treatment.

### Determining Amount of 6-TG and 6-MMP Nucleotide Compounds in a Sample

In another aspect, the amounts of the one or more 6-thioguanine nucleotide compounds and the one or more 6-methylmercaptopurine nucleotide compounds can be determined concurrently. Thus, in some embodiments, the invention provides methods for determining the amount of thiopurine drug metabolites in a sample, the methods comprising: (a) providing a sample comprising one or more 6-thioguanine nucleotide compounds and one or more 6-methylmercaptopurine nucleotide compounds; (b1) converting the one or more 6-thioguanine nucleotide compounds to 6-thioguanine or a 6-thioguanine hydrolysis product, and (b2) converting the one or more 6-methyl-mercaptopurine nucleotide compounds to 6-methylmercaptopurine or a 6-methylmercaptopurine hydrolysis product; (c1) chromatographically separating the 6-thioguanine or the 6-thioguanine hydrolysis product from other components in the converted sample using liquid chromatography, and (c2) chromatographically separating the 6-methylmercaptopurine or the 6-methylmercaptopurine hydrolysis product from other components in the converted sample using liquid chromatography; (d1) analyzing the chromatographically separated 6-thioguanine or the 6-thioguanine hydrolysis product by mass spectrometry to determine the amount of the one or more 6-thioguanine nucleotide compounds in the sample, and (d2) analyzing the chromatographically separated 6-methylmercaptopurine or the 6-methylmercaptopurine hydrolysis product by mass spectrometry to determine the amount of the one or more 6-methylmercaptopurine nucleotide compounds in the sample. In some embodiments, the sample originated from a biological fluid, such as whole blood.

Additional embodiments of such methods include any of the embodiments described above for the methods of determining the amounts of the one or more 6-thioguanine nucleotide compounds in a sample and the methods for determining the amounts of the one or more 6-methylmercaptupurine nucleotide compounds in a sample. Modification of the above methods to permit concurrent analysis can be determined by those of skill in the art.

Systems for Analyzing 6-TG and/or 6-MMP Nucleotide Compounds in a Sample

In another aspect, the invention provides systems for carrying out any of the aforementioned methods of analysis. Thus, in at least one aspect, the invention provides systems for determining the amount of thiopurine drug metabolites in a sample, the systems comprising: (a) a sample comprising one or more 6-thioguanine nucleotide compounds and/or one or more 6-methylmercaptopurine nucleotide compounds; (b) a station for converting the one or more 6-thioguanine nucleotide compounds to 6-thioguanine or a 6-thioguanine hydrolysis product and/or for converting the one or more 6-methyl-mercaptopurine nucleotide compounds to 6-methylmercaptopurine or a 6-methylmercaptopurine hydrolysis product; (c) a station for chromatographically separating the 6-thioguanine or the 6-thioguanine hydrolysis product from other components in the converted sample using liquid chromatography, and/or for chromatographically separating the 6-methylmercaptopurine or the 6-methylmercaptopurine hydrolysis product from other components in the converted sample using liquid chromatography; and (d) a station for analyzing the chromatographically separated 6-thioguanine or the 6-thioguanine hydrolysis product by mass spectrometry to determine the amount of the one or more 6-thioguanine nucleotide compounds in the sample, and/or for analyzing the chromatographically separated 6-methylmercaptopurine or the 6-methylmercaptopurine hydrolysis product by mass spectrometry to determine the amount of the one or more 6-methylmercaptopurine nucleotide compounds in the sample. In some embodiments, the system further comprises a station for preparing the sample from a biological fluid, such as whole blood.

Such systems can include various embodiments and subembodiments analogous to those described above for methods of analyzing samples comprising one or more 6-thioguanine nucleotide compounds and/or one or more 6-methylmercaptopurine nucleotide compounds.

These systems include various stations. As used herein, the term “station” is broadly defined and includes any suitable apparatus or collections of apparatuses suitable for carrying out the recited method. The stations need not be integrally connected or situated with respect to each other in any particular way. The invention includes any suitable arrangements of the stations with respect to each other. For example, the stations need not even be in the same room. But in some embodiments, the stations are connected to each other in an integral unit.

### EXAMPLE

### Example 1

The following test protocol is exemplary and additional or other analytical methods and equipment can be used as long as they do not depart from the methodology disclosed herein. The thiopurine metabolites, 6-thioguanine nucleotides (6-TGNs) and 6-methylmercaptopurine nucleotides (6-MMPNs), were measured by mass spectrometric detection following washed red blood cell lysis, precipitation, hydrolysis and chromatographic separation. Patient samples were received and immediately washed and resuspended in buffer. Red blood cell (RBC) counts were obtained for a portion of each patient sample. Stable labeled isotope was added as an internal standard to an aliquot of the second (lysed) portion of the sample which was then mixed with dithiothreitol (DTT) to prevent thiol oxidation and perchloric acid to precipitate protein and hydrolyze the nucleotides. Supernatant was aliquoted and sample was hydrolyzed to produce the analytes of interest. Buffer was added and the samples were mixed and then injected onto the LC-MS/MS system (Aria Transcend TX4 LC). An MDS-Sciex API5000 triple quadrupole mass spectrometer, operating in positive ion electrospray ionization mode was used for detection.

Quantification of analyte and internal standards was performed in multiple reaction monitoring mode (MRM). The back-calculated amount of the compounds in each sample was determined from calibration curves generated by spiking known amounts of purified analyte into defibrinated plasma from 0.1-10.0 picomoles per microliter (pmol/μL) for 6-TG and 0.5-50 pmol/μL for 6-MMP.

Sample Red Blood Cell Washing Protocol

Tubes of whole blood and a bottle of Coombscell-E were placed on a tube rocker to mix for at least 15 minutes. Aliquots (500 μL) of each whole blood sample and negative control were pipetted into separate labeled 12×75mm polypropylene tubes. Isotonic saline (4 mL, 0.9% w/v) was then added to each tube and the tubes were loaded into the rotor of a cell washer. The tubes were agitated and spun on the cell washer for about 3 minutes. After the wash cycle on the cell washer, plasma and saline were removed from each tube by pipette. The washing protocol (addition of isotonic saline and spinning/agitation) was performed three times for a total of four (4) wash cycles.

After removal of saline from the tubes, 650 μL of Cellpack buffer was added to each tube. The tubes were capped and placed on a tube rocker to resuspend the cells thoroughly. The suspensions are now ready for analysis.

6-Methylmercaptopurine (6-MMP)/6-thioguanine (6-TG) Extraction

Aliquots (100 μL) of lysed samples, standards, and controls were pipetted into 12×75 mm glass tubes. To each of the sample tubes 150 μL of internal standard and 150 μL 0.4M DTT to each tube were added. Tubes containing control (double blanks) received 150 μL 0.4M DTT and 150 μL of deionozied water. Perchloric acid (40 μL, 70% in water) was added to each tube. The tubes were vortexed for about 15 seconds at 2500 rpm and centrifuged for about 5 minutes at 3500 rpm.

A 250 μL sample of acidic supernatant was added to a clean 13×100 mm glass tube, which was placed into a boiling water bath for about 2 hours and subsequently allowed to cool. An 100 μL aliquot was pipetted from each of the 13×100 mm glass tubes into a 96-well plate, followed by addition of 133 μL of 1M ammonium acetate to each well. The plate was vortexed for about 1 minute at 2500 rpm and centrifuged for about 5 minutes at 3500 rpm. After centrifugation, 20 μL samples were injected into an LC/MS system as set forth herein.

### Mass Spectrophotometry Analysis

Transition ratio assessment is performed by dividing the area response of the qualifying transition by the area response of the quantifying transition compared to the average transition ratio of the calibrators prepared in the same batch. Table 1 lists quantify and qualifying transition ranges based on the analyte. The designation “IS” in Table 1 means internal standard.

TABLE 1

| Internal Standard/Analyte | Quantifying Transition | Qualifying Transition |
|---|---|---|
| 6-MMP Derivative IS | 161.1/110.1 | N/A |
| 6-TG IS | 171.0/154.0 | N/A |
| 6-MMP Derivative | 158.1/110.1 | 158.1/82.1 |
| 6-TG | *167.9/151.0, | *167.9/133.9, |
| | 167.9/151.1 | 167.9/133.8, |
| | | 167.9/134.0 |

### REPORTED REFERENCE INTERVAL

TABLE 2

| | Therapeutic Range | Alert Values |
|---|---|---|
| Analyte | (pmol/8 × 10⁸RBC) | (pmol/8 × 10⁸RBC) |
| 6-TGNs | 235 - 450 | >450 |
| 6-MMPNs | <5700 | >5700 |

Table 2 lists exemplary therapeutic levels for 6-thioguanine nucleotides (6-TGNs) and 6-methylmercaptopurine nucleotides (6-MMPNs) and alert levels for these nucleotides. Nucleotide levels in the alert value range require_{—} modulation of the nucleotide therapy in order to reduce the corresponding levels in the blood.

We claim:

1. A method for determining the amount of thiopurine drug metabolites in a sample, the method comprising:
(a) providing a sample comprising one or more 6-thioguanine nucleotide compounds and one or more 6-methylmercaptopurine nucleotide compounds;
(b1) converting the one or more 6-thioguanine nucleotide compounds to 6-thioguanine or a 6-thioguanine hydrolysis product, and (b2) converting the one or more 6-methyl-mercaptopurine nucleotide compounds to 6-methylmercaptopurine or a 6-methylmercaptopurine hydrolysis product;
(c1) chromatographically separating the 6-thioguanine or the 6-thioguanine hydrolysis product from other components in the converted sample using liquid chromatography, and (c2) chromatographically separating the 6-methylmercaptopurine or the 6-methylmercaptopurine hydrolysis product from other components in the converted sample using liquid chromatography;
(d1) analyzing the chromatographically separated 6-thioguanine or the 6-thioguanine hydrolysis product by mass spectrometry to determine the amount of the one or more 6-thioguanine nucleotide compounds in the sample, and (d2) analyzing the chromatographically separated 6-methylmercaptopurine or the 6-methylmercaptopurine hydrolysis product by mass spectrometry to determine the amount of the one or more 6-thioguanine nucleotide compounds in the sample.

2. The method of claim 1, wherein the 6-methylmercaptopurine hydrolysis product is 4-amino-5-(methylthio)carbonyl imidazole.

3. The method of claim 1, wherein the converting step (b1) or (b2) comprises contacting the one or more 6-thioguanine nucleotide compounds with a sulfur-containing reducing agent to produce one or more reduced 6-thioguanine nucleotide compounds or contacting the one or more 6-methylmercaptopurine nucleotide compounds with a sulfur-containing reducing agent to produce one or more reduced 6-methylmercaptopurine nucleotide compounds.

4. The method of claim 3 wherein the sulfur-containing reducing agent is dithioerythritol.

5. The method of claim 3, wherein the converting step (b1) comprises contacting the one or more reduced 6-thioguanine nucleotide compounds with an acidic hydrolyzing agent to produce 6-thioguanine or the 6-thioguanine hydrolysis product.

6. The method of claim 5, wherein the converting step (b1) produces a mixture of 6-thioguanine and the 6-thioguanine hydrolysis product.

7. The method of claim 6, wherein the molar ratio of the 6-thioguanine hydrolysis product to 6-thioguanine in the mixture is at least 2:1, or at least 3:1, or at least 4:1, or at least 5:1, or at least 10:1, or at least 25:1, or at least 100:1.

8. The method of claim 3, wherein the converting step (b2) comprises contacting the one or more reduced 6-methylmercaptopurine nucleotide compounds with an acidic hydrolyzing agent to produce 6-methylmercaptopurine or the 6-methylmercaptopurine hydrolysis product.

9. The method of claim 8, wherein the converting step (b2) produces a mixture of 6-methylmercaptopurine and the 6-methylmercaptopurine hydrolysis product.

10. The method of claim 9, wherein the molar ratio of the 6-methylmercaptopurine hydrolysis product to 6-methylmercaptopurine in the mixture is at least 2:1, or at least 3:1, or at least 4:1, or at least 5:1, or at least 10:1, or at least 25:1, or at least 100:1.

11. The method of claim 5, wherein the acidic hydrolyzing agent is perchloric acid, nitric acid, sulfuric acid, or a combination thereof.

12. The method of claim 8, wherein the acidic hydrolyzing agent is perchloric acid, nitric acid, sulfuric acid, or a combination thereof.

13. The method of claim 1, wherein the converting step(s) (b1) and (b2) are carried out in an automated fashion.

14. The method of claim 1, wherein the converting step(s) (b1) and (b2) are carried out manually.

15. The method of claim 1, wherein using liquid chromatography comprises using analytical liquid chromatography.

16. The method of claim 1, wherein the analyzing step (d1) comprises ionizing the 6-thioguanine or the 6-thioguanine hydrolysis product.

17. The method of claim 1, wherein the analyzing step (d2) comprises ionizing the 6-methylmercaptopurine or the 6-methylmercaptopurine hydrolysis product.

18. The method of claim 1, wherein the analyzing step (d1) comprises detecting the 6-thioguanine or the 6-thioguanine hydrolysis product using a mass spectrometer.

19. The method of claim 1, wherein the analyzing step (d2) comprises detecting the 6-methylmercaptopurine or the 6-methylmercaptopurine hydrolysis product using a mass spectrometer.

20. The method of claim 1, where the analyzing step (d1) includes determining the amount of the one or more 6-thioguanine nucleotide compounds in the sample, and the analyzing step (d2) includes determining the amount of the one or more 6-methylmercaptopurine nucleotide compounds in the sample.

21. The method of claim 1, wherein the sample comprises one or more internal standards.

22. The method of claim 21, wherein the one or more internal standards comprise stable isotopically-labeled forms of 6-thioguanine, the 6-thioguanine hydrolysis product, 6-methylmercaptopurine, or the 6-methylmercaptopurine hydrolysis product.

23. The method of claim 22, wherein the stable isotopically-labeled forms include deuterium, carbon-13, nitrogen-15, sulfur-34, or any combination thereof.

24. The method of claim 1, wherein the sample originated from a biological sample.

25. The method of claim 24, wherein the biological sample is whole blood, plasma, serum, urine, cerebrospinal fluid, tissue homogenate, saliva, amniotic fluid, bile, mucus, peritoneal fluid, or lymphatic fluid.

26. The method of claim 25, wherein the biological sample is whole blood.

27. The method of claim 26, further comprising determining a relative amount of the one or more 6-thioguanine nucleotide compounds and the one or more 6-methylmercaptopurine nucleotide compounds to the number of red blood cells in the biological sample.

28. The method of claim 27, further comprising correlating the relative amount of the one or more 6-thioguanine nucleotide compounds in the biological sample to the efficacy of thiopurine treatment of a human subject.

29. The method of claim 27, further comprising correlating the relative amount of the one or more 6-methylmercaptopurine nucleotide compounds to an increased risk of hepatotoxicity in a human subject undergoing thiopurine treatment.

30. The method of claim 27, further comprising correlating the relative amount of the one or more 6-thioguanine nucleotide compounds to an increased risk of leucopenia in a human subject undergoing thiopurine treatment.

31. A method for determining the amount of thiopurine drug metabolites in a sample, the method comprising:
(a) providing a sample comprising one or more 6-thioguanine nucleotide compounds;
(b) converting the one or more 6-thioguanine nucleotide compounds to 6-thioguanine or a 6-thioguanine hydrolysis product;
(c) chromatographically separating the 6-thioguanine or the 6-thioguanine hydrolysis product from other components in the converted sample using liquid chromatography;
(d) analyzing the chromatographically separated 6-thioguanine or the 6-thioguanine hydrolysis product by mass spectrometry to determine the amount of the one or more 6-thioguanine nucleotide compounds in the sample.

32. A method for determining the amount of thiopurine drug metabolites in a sample, the method comprising:
(a) providing a sample comprising one or more 6-methylmercaptopurine nucleotide compounds;
(b) converting the one or more 6-methylmercaptopurine nucleotide compounds to 6-methylmercaptopurine or a 6-methylmercaptopurine hydrolysis product;
(c) chromatographically separating the 6-methylmercaptopurine or the 6-methylmercaptopurine hydrolysis product from other components in the converted sample using liquid chromatography;
(d) analyzing the chromatographically separated 6-methylmercaptopurine or the 6-methylmercaptopurine hydrolysis product by mass spectrometry to determine the amount of the one or more 6-methylmercaptopurine nucleotide compounds in the sample.

33. A system for determining the amount of thiopurine drug metabolites in a sample, the systems comprising:
(a) a sample comprising one or more 6-thioguanine nucleotide compounds and one or more 6-methylmercaptopurine nucleotide compounds;
(b) a station for converting the one or more 6-thioguanine nucleotide compounds to 6-thioguanine or a 6-thioguanine hydrolysis product and for converting the one or more 6-methylmercaptopurine nucleotide compounds to 6-methylmercaptopurine or a 6-methylmercaptopurine hydrolysis product;
(c) a station for chromatographically separating the 6-thioguanine or the 6-thioguanine hydrolysis product from other components in the converted sample using liquid chromatography, and for chromatographically separating the 6-methylmercaptopurine or the 6-methylmercaptopurine hydrolysis product from other components in the converted sample using liquid chromatography; and
(d) a station for analyzing the chromatographically separated 6-thioguanine or the 6-thioguanine hydrolysis product by mass spectrometry to determine the amount of the one or more 6-thioguanine nucleotide compounds in the sample, and for analyzing the chromatographically separated 6-methylmercaptopurine or the 6-methylmercaptopurine hydrolysis product by mass spectrometry to determine the amount of the one or more 6-methylmercaptopurine nucleotide compounds in the sample.
